(19) United States
(12) Patent Application Publication
(10) Pub. No.: US 2003/0124068 A1
(43) Pub. Date: Jul. 3, 2003

(54) Oral preparation

(76) Inventors: Yamagishi Atsushi, Tokyo (JP); Yoshinori Murakami, Tokyo (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/332,980

(22) PCT Filed: Jul. 24, 2001
(86) PCT No.: PCT/JP01/06373

(30) Foreign Application Priority Data
Jul. 24, 2000 (JP) 2000-222707

(51) Int. Cl. A61K 7/18

(52) U.S. Cl. 424/52

(57) ABSTRACT

Provided is an oral preparation which can easily give white, smooth and glossy teeth. The oral preparation comprises the following components (A), (B) and (C):

(A) 0.02 to 0.7 wt. % (in terms of fluorine atom) of a fluoride compound,

(B) 0.1 to 5 mol/kg of an acid compound having a pKa (25° C.) of 2.5 to 6.0 or salt thereof, and

(C) 5 to 90 wt. % of water, having a pH, as the preparation itself or a 30 wt. % aqueous solution of the preparation, ranging from 3 to 3.5.

### TECHNICAL FIELD

The present invention relates to an oral preparation having whitening action when applied to the surface of the teeth, more specifically, an oral preparation capable of imparting whiteness and gloss to the surface of the teeth.

### BACKGROUND ART

Discoloring of the teeth occurs owing to external factors such as adhesion of a colored substance to the tooth surface due to tartar, plaque, smoking or daily drinking of coffee or tea; and internal factors such as aging which causes discoloration of the dentin appearing through the enamel having high transparency and use of a medicament such as tetracycline during the enamel formative years, which use discolors the enamel itself. In order to fundamentally whiten the teeth, it is therefore necessary to take countermeasures against not only external factors but also internal factors.

A variety of physical or chemical methods have heretofore been reported as means for whitening the teeth. Reported as physical methods are, in addition to removal of the colored substance by polishing, a method of removing by using n-butyl ether or butyl butylate (Japanese Patent Laid-Open No. Hei 1-203316, Japanese Patent Laid-Open No. Hei 1-104004) and a method of improving the tooth color by covering the teeth with ceramic veneers. As the chemical methods, known are a method of promoting remineralization by an oral composition containing hydroxyapatite (Japanese Patent Laid-Open No. Hei 1-305020, Japanese Patent Laid-Open No. Hei 9-202718) and a method of oxidation bleaching with a peroxide (Japanese Patent Publication No. Hei 6-8248). Recently, a teeth whitening composition obtained by adding, to a peroxide, a self curing type calcium phosphate compound and a fluorine compound is reported (Japanese Patent Laid-Open No. Hei 11-116421).

The conventional methods are however still insufficient in the teeth whitening effects or involve some problems. Application of ceramic veneers needs, prior thereto, buffing of the tooth surface and use of veneers requires the treatment and guidance by dentists. Such dental treatment by experts inevitably costs high.

An object of the present invention is therefore to provide an oral preparation capable of easily whitening the teeth in daily life without the guidance or treatment by dentists.

### DISCLOSURE OF THE INVENTION

The present inventors have found that by supplying fluoride ion in a buffer system maintained at a predetermined pH, the teeth become white, smooth and glossy without the treatment by an expert. They have also revealed that under such conditions, gradual formation of calcium fluoride on the surface or surface layer of the teeth can be observed. Based on such a finding, they have carried out an extensive investigation on a composition capable of forming calcium fluoride in the oral cavity and continuously supplying it onto the tooth surface. As a result, it has been found that the teeth can be whitened by a preparation containing, in combination, a fluoride ion supplying component and an acid compound having a predetermined hydrogen ion dissociation constant or salt thereof and having a pH, as that of the preparation itself or a 30 wt. % aqueous solution thereof, adjusted to 3 to 5.5. The above-described conditions coincide with those for efficiently forming calcium fluoride on the tooth surface, when the above-described preparation is applied to the oral cavity, by keeping a pH so as to permit at least a predetermined hydroxyapatite- or fluorapatite-dissolving capacity and a markedly low calcium-fluoride-dissolving capacity. Formation of such calcium fluoride is believed to lead to excellent whitening effects of the teeth.

In the present invention, there is thus provided an oral preparation comprising the following components (A), (B) and (C):

(A) 0.02 to 0.7 wt. % (in terms of fluorine atom) of a fluorine ion supplying component,

(B) 0.1 to 5 mol/kg of an acid compound having a pKa (25° C.) of 2.5 to 6.0 or salt thereof, and

(C) 5 to 90 wt. % of water, and having a pH, as that of the preparation itself or a 30 wt. % of an aqueous solution thereof, ranging from 3 to 5.5.

### THE BEST MODE FOR CARRYING OUT THE INVENTION

No particular limitation is imposed on the fluorine ion supplying component as Component (A) insofar as it is usable in the oral cavity. Examples include inorganic fluorides such as sodium fluoride, potassium fluoride, ammonium fluoride and lithium fluoride and organic fluorides such as amine fluorides. Of these, sodium fluoride and ammonium fluoride are preferred from the viewpoints of safety, solubility and taste.

When fluoride ions are supplied by sodium fluoride, a calcium salt or zinc salt which has already existed in the preparation reacts with them and the supply amount of fluoride ions lowers. Incorporation of such a multivalent metal salt is therefore not preferred in the present invention.

In the oral preparation of the present invention, one or more than one of these fluoride compounds can be incorporated.

The content of the fluoride ion supplying component (A) preferably ranges from 0.02 to 0.7 wt. % (which will hereinafter be called “%” simply), in terms of a fluorine atom contained in the fluorine ion supplying component, based on the whole preparation of the present invention from the viewpoints of whitening effects of teeth and reinforcement of teeth quality. Particularly for home use, its content is preferably suppressed to a range of 0.02 to 0.2% in order to avoid appearance of its toxicity due to accidental ingestion.

The acid compound as Component (B) has a pKa (25° C.) of from 2.5 to 6.0, more preferably from 2.5 to 5.0, still more preferably from 3 to 4.5. Within the above-described range, a preparation having a desired pH which permits sufficient buffer action, progress in efficient formation of calcium fluoride, sufficient whitening effects, and appropriate hydroxyapatite- or fluorapatite-dissolving capacity is available so that there is no fear of it damaging the teeth. It is to be noted that the pKa is the negative logarithm of an acid dissociation constant (described, for example, in “Kagaku Binran Kisohen, Revised 2nd Edition”, p. 993, published by Maruzen Co., Ltd., Sixth Impression published on Sep. 20, 1981; “Kagaku Binran Kisohen, Revised 4th Edition”, p. 317, published by Maruzen Co., Ltd., published on Sep. 30, 1993).

Examples of such an acid compound include monobasic acids such as formic acid, acetic acid and propionic acid; dibasic acids such as oxalic acid, succinic acid, fumaric acid and maleic acid; hydroxycarboxylic acids such as lactic acid, glycolic acid, tartaric acid, malic acid, citric acid and ascorbic acid; acidic amino acids such as glutamic acid and aspartic acid; keto acids such as levulinic acid; and aromatic carboxylic acids such as benzoic acid and salicylic acid.

Of these, at least one acid selected from lactic acid, acetic acid, citric acid, malic acid, succinic acid, tartaric acid and adipic acid is especially preferred.

Examples of the salt of the acid compound include alkali metal salts such as sodium salts and potassium salts. Upon production of the preparation of the present invention, the salt of such an acid compound can be added, or alternatively, the acid compound and an alkali are added separately and a buffer system of the acid compound and/or salt thereof may be formed in the preparation. As the alkali, sodium hydroxide and potassium hydroxide are typical examples but no limitation is imposed insofar as it neutralizes the acid and accelerates ionization of the acid existing in the acid form.

The acid compound and/or salt thereof as Component (B) is preferably incorporated in the preparation of the present invention in an amount, as the total of the acid compound and the salt, in short, as the total of the acid and salt, of from 0.1 to 5 mol/kg, especially from 0.1 to 2 mol/kg, in order to attain whitening effects. To impart them with a buffering capacity, the molar ratio of the acid to salt is preferably adjusted to from 10:1 to 1:10.

The oral preparation of the present invention must be, as described above, substantially free of multivalent metal ions such as magnesium, zinc and calcium for maintaining its effects. Unless fluoride ions can be supplied quickly, calcium fluoride cannot be formed under acidic conditions, which causes elution of calcium from the teeth, whereby the teeth become white but lose gloss. There is therefore a fear of the preparation containing such a multivalent metal ion adversely affecting the teeth by its long use. The fact that the oral preparation of the present invention contains a buffer system also means that the preparation contains water as Component (C). The preparation of the present invention preferably has a water content of from 5 to 90%. It is essentially necessary that the preparation must be in the form of an aqueous solution for exhibiting a buffering performance. Existence of water is also necessary for immediate supply of fluorine ions.

It is important that the oral preparation of the present invention itself or in the form of a 30% aqueous solution has a pH of from 3 to 5.5 in order to, when it is applied to the oral cavity, impart it with hydroxyapatite- or fiuorapatite-dissolving capacity and suppress calcium-fluoride dissolving capacity at a low level. The pH is more preferably from 3.5 to 5.0. Upon measurement of pH, the concentration of the preparation is selected as needed, depending on the using state of the oral preparation of the present invention. For example, when it is used as a toothpaste, the measurement is conducted at the actual using concentration supposed to be 30%. When it is used as a mouth wash, the pH of the preparation itself is measured without dilution.

It is preferred to incorporate an anionic surfactant (D) in the oral preparation of the present invention in order to heighten the whitening effect of teeth. As the anionic surfactant, preferred are higher akyl sulfate ester salts, N-alkyl sarcosinate salts, and higher fatty acid monoglyceride monosulfate salts. The alkyl group or fatty acid residue of these surfactants has preferably 8 to 24, especially 8 to 18 carbon atoms. As the salt of these surfactants, alkali metal salts, ammonium salts and organic amine salts are preferred. The surfactant is preferably incorporated in the preparation of the present invention in an amount of from 0.1 to 5%, especially from 0.2 to 2% from the viewpoint of the whitening effects of the teeth.

In addition to the above-described components, the oral preparation of the present invention can contain an effervescent agent, an effervescent assistant, abrasive, humectant, binder, extender, sweetener, preservative, sterilizer, medicinal component, adhesive, pigment, colorant and/or perfume as needed. Polyethylene glycol, which is a conventional whitening component, can also be used in combination.

The preparation of the present invention can take any one of solution, gel and paste forms. In any form, polyethylene glycol, propylene glycol, glycerin, sorbitol, maltitol, xylitol, lactitol or erythritol can be added as a humectant or thickener.

As a thickener of the solution preparation, as a gelatinizer for the gel preparation, or as a binder for the paste preparation, it is possible to add carboxymethylcellulose sodium, hydroxyethyl cellulose, carboxyvinyl polymer, xanthan gum, carrageenan, sodium alginate, hydroxypropyl cellulose, guar gum, sodium chondroitin sulfate or the like. In the case where the preparation has a high concentration owing to the buffer system, addition of a nonionic polymer such as hydroxyethyl cellulose, guar gum or hydroxypropyl cellulose is preferred.

The oral preparation of the present invention can be prepared in a conventional manner. A toothpaste can be prepared, for example, by weighing a prescribed amount of each component such as purified water, humectant, binder, flavor, preservative, sweetener, buffer component, fluorine ion supplying component, and medicinal component, and if necessary another medicinal component, mixing them under predetermined conditions to swell the binder, adding to the mixture an abrasive and effervescent, and mixing them while degassing. The pH adjustment may be conducted after preparation if necessary.

The oral preparation of the present invention thus obtained dissolves hydroxyapatite or fluorapatite so that it dissolves colored hydroxyapatite on the tooth surface. On the other hand, it does not dissolve calcium fluoride so much so that fluorine ions react with calcium ions in the saliva, thereby efficiently forming calcium fluoride on the tooth surface. The calcium fluoride layer thus formed on the tooth surface has acid resistance, thereby preventing release of calcium ions or phosphoric acid ions from the tooth surface. The calcium fluoride layer is not peeled or scraped off by the mechanical action such as brushing by a toothbrush and its retention on the tooth is excellent. Accordingly, the oral preparation of the present invention is useful for whitening the teeth, because white, smooth and glossy teeth are easily available by the application of it. As described later in Examples, use of the invention product alleviates the pain of the subject suffering from hyperesthesia which otherwise occurs owing to drinking of cold water or the like. It is therefore also effective for hyperesthesia. Judging from a large intake amount of fluoride, it will be effective also for prevention of caries.

### EXAMPLE 1

Using acids different in pKa, 1M aqueous solutions containing 23% sodium fluoride were prepared, followed by adjustment to pH 4 with sodium hydroxide. After measuring their dissolving capacity, a cow tooth (having a mirror polished surface), which had been photographed in advance, was immersed in each solution for 24 hours. After the treatment, the tooth was taken out from the solution and photographed again. The color of the tooth before immersion was compared with that after immersion. The comparison was made as follows: A panel of 20 experts was asked to see the photographs of the tooth before and after treatment and to evaluate it as ◯ when the teeth was whitened after the treatment, while as × when no change in color was recognized. Upon evaluation, the tooth which was whitened but lost its gloss was evaluated as ×. The preparation evaluated as ◯ by at least a half of the experts was judged effective and the others were judged ineffective.

Dissolving capacity was measured in the following manner. Described specifically, the preparation is diluted to 30 wt. % with deionized water. The phosphorus and calcium concentrations in the solution were each determined in advance by using a calorimetric reagent and they were subtracted later. To 100 ml of the diluted solution were added hydroxyapatite (HAP), fluorapatite (FAP) and CAF₂, each in the powdery form. The resulting mixture was stirred for 24 hours by a magnetic stirrer and the reaction mixture was filtered to remove the solid matter. The phosphorus and calcium concentrations in the solution were determined by a calorimetric reagent.

With regards to the HAP- or FAP-dissolving capacity, the preparation capable of satisfying either one of the following standards: 200 mg/L or greater of calcium and 300 mg/L or greater of phosphorus is evaluated as ◯. With regards to the CaF₂-dissolving capacity, the preparation capable of satisfying the following standard, 150 mg/L or less of calcium, is evaluated as ◯. In Table 1, the preparation which has satisfied both standards is indicated as ◯. As the calorimetric reagents for calcium and phosphorus, Calcium Test Wako (for determination of calcium, product of Wako Pure Chemicals) and P Test Wako (for determination of inorganic phosphorus, product of Wako Pure Chemicals) were employed, respectively.

As a result, it has been found that a preparation obtained by adding an acid having a pKa of from 2.5 to 6.0 to 0.02 to 0.7% of sodium fluoride, and adding an alkali to the resulting mixture to adjust its pH to 3 to 5.5 exhibits good hydroxyapatite- or fluorapatite-dissolving capacity but poor calcium-fluoride-dissolving capacity and has excellent teeth whitening effects.

TABLE 1

| | | | Dissolving | Whitening |
|---|---|---|---|---|
| Acid added | | pKa | capacity | effect |
| Comp. Ex. | Hydrochloric acid | −8 | ∘ | x |
| | Sulfuric acid | 1.99 | ∘ | x |
| | Phosphoric acid | 2.15 | ∘ | x |
| Example | Citric acid | 2.9 | ∘ | ∘ |
| | Tartaric acid | 2.99 | ∘ | ∘ |
| | Malic acid | 3.24 | ∘ | ∘ |
| | Lactic acid | 3.66 | ∘ | ∘ |
| | Acetic acid | 4.56 | ∘ | ∘ |

### EXAMPLE 2

Aqueous solutions shown in Table 2 were prepared and as in Example 1, their dissolving capacity and whitening effect were evaluated. As a result, it has been found that addition of an anionic surfactant enhances the whitening effect.

TABLE 2
(%)

| | | | | Comp. |
|---|---|---|---|---|
| | Examples | | | Ex. |
| Component | 2-1 | 2-2 | 2-3 | 2-1 |
| Sodium lauryl sulfate | 1 | | 1 | |
| Sodium N-myristoyl sarcosinate | | 1 | | |
| Phosphoric acid | | | | 5 |
| Citric acid | 10 | | | |
| Tartaric acid | | 5 | | |
| Malic acid | | | 5 | |
| Lactic acid | | | | |
| Sodium fluoride | 0.3 | 0.1 | 0.21 | 2 |
| Sodium hydroxide | q.s. | q.s. | q.s. | q.s. |
| Water | Bal- | Bal- | Bal- | Bal- |
| | ance | ance | ance | ance |
| Ph | 3.5 | 4 | 4.5 | 4 |
| Dissolving capacity | ∘ | ∘ | ∘ | ∘ |
| Whitening effect | ∘ | ∘ | ∘ | x |

### EXAMPLE 3

Toothpastes as shown in Table 3 were prepared in a conventional manner. Dissolving capacity of each of their 30% aqueous solutions was measured as in Example 1. Their whitening effect was measured also as in Example 1 (by immersing in the toothpaste for 24 hours).

Ten subjects aware of their hyperesthesia were asked to use the toothpastes shown in Table 3 (Examples 3-1 to 3-4) for one month instead of their daily used ones. As a result, all the subjects who used the toothpastes of Examples 3-1 to 3-4 evaluated that the degree of hyperesthesia (the degree of pain which was sensed upon drinking cold water) was alleviated.

TABLE 3
(%)

| | Examples | | | | | Comp. Ex. | | |
|---|---|---|---|---|---|---|---|---|
| | 3-1 | 3-2 | 3-3 | 3-4 | 3-5 | 3-1 | 3-2 | 3-3 |
| | Dental | Dental | Dental | Dental | Mouth | Dental | Dental | Mouth |
| Components | paste | paste | paste | paste | wash | paste | paste | wash |
| Lactic acid | 5 | | | 5 | 3 | 7 | | |
| Citric acid | | 10 | | 5 | | | | |
| Malic acid | | | 10 | | | | | |
| Hydrochloric acid | | | | | | | 2 | |
| 70% solution of sorbitol | 35 | 35 | 35 | 35 | 10 | 35 | 1 | 10 |
| Propylene glycol | 5 | 5 | 5 | 5 | | 5 | 5 | |
| Methyl p-oxybenzoate | 0.1 | 0.1 | 0.1 | 0.1 | 0.1 | 0.1 | 0.1 | 0.1 |
| Saccharin sodium | 0.1 | 0.1 | 0.1 | 0.1 | | 0.1 | 0.1 | |
| Sodium lauryl sulfate | 1 | | | 1 | | 1 | | |
| Polyoxyethylene hydrogenated castor oil | | 1 | 1 | | 0.5 | | 1 | |
| Sodium fluoride | 0.21 | 0.21 | 0.21 | 0.21 | 0.042 | 0.21 | 0.21 | 0.042 |
| Hydroxyethyl cellulose | 0.5 | 0.5 | 0.5 | 0.5 | | 0.5 | 0.5 | |
| Carboxymethyl cellulose | 0.5 | 0.5 | 0.5 | 0.5 | | 0.5 | 0.5 | |
| Hydrous silicic acid | 15 | 15 | 15 | 15 | | 15 | 15 | |
| Perfume | 1 | 1 | 1 | 1 | 0.2 | 1 | 1 | |
| pH regulator (NaOH, HCl) | q.s. | q.s. | q.s. | q.s. | q.s. | q.s. | q.s. | q.s. |
| Water | Bala- | Bala- | Bala- | Bala- | Bala- | Bala- | Bala- | Bala- |
| | nce | nce | nce | nce | nce | nce | nce | nce |
| PH | 4 | 4 | 4 | 4.5 | 4.5 | 7 | 4 | 7 |
| Dissolving capacity | ◯ | ◯ | ◯ | ◯ | ◯ | X | ◯ | X |
| Whitening effect | ◯ | ◯ | ◯ | ◯ | ◯ | X | X | X |

### INDUSTRIAL APPLICABILITY

Use of the oral preparation of the present invention makes it possible to readily obtain white, smooth and glossy teeth.

1. An oral preparation comprising the following components (A), (B) and (C):
(A) 0.02 to 0.7 wt. % (in terms of fluorine atom) of a fluoride ion supplying component,
(B) 0.1 to 5 mol/kg of an acid compound having a pKa (25° C.) of 2.5 to 6.0 or salt thereof, and
(C) 5 to 90 wt. % of water, and having a pH, as the preparation itself or a 30 wt. % aqueous solution of the preparation, ranging from 3 to 3.5.

2. An oral preparation of claim 1, further comprising (D) an anionic surfactant.

3. An oral preparation of claim 1 or 2, wherein the acid as Component (B) is at least one selected from lactic acid, acetic acid, citric acid, malic acid, succinic acid, tartaric acid and adipic acid.
